(19) United States
(12) Patent Application Publication
(10) Pub. No.: US 2015/0150888 A1
(43) Pub. Date: Jun. 4, 2015

(54) COMBINATION OF SELECTIVE PROGESTERONE-RECEPTOR MODULATORS AND NONSTEROIDAL ANTI-INFLAMMATORY DRUGS

(71) Applicant: LABORATOIRE HRA-PHARMA, Paris (FR)

(72) Inventors: Michele Resche-Rigon, Paris (FR); Delphine Levy, Bagnolet (FR); Erin Gainer, Paris (FR)

(21) Appl. No.: 14/380,867

(22) PCT Filed: Feb. 28, 2013
(86) PCT No.: PCT/FR2013/050424

§ 371 Date: Aug. 25, 2014

(30) Foreign Application Priority Data
Feb. 28, 2012 (FR) 1251771

(51) Int. Cl. A61K 31/57; A61K 31/192; A61K 31/5415

(52) U.S. Cl. CPC A61K 31/57; A61K 31/5415; A61K 31/192

(57) ABSTRACT

The invention relates to a pharmaceutical composition combining a selective progesterone receptor modulator (SPRM), such as ulipristal acetate, and one or more non-steroidal anti-inflammatory compounds, in particular in a method of regular contraception, or in emergency or on-demand contraception.

The present invention relates to a method of contraception.

### TECHNOLOGICAL BACKGROUND

Ulipristal acetate is a selective progesterone receptor modulator which inhibits or delays ovulation (Stratton et al., Human Reproduction, 2000, 15(5): 1092-1099). Ulipristal acetate is the active ingredient of the EllaOne® pill marketed for emergency contraception.

Emergency contraception makes it possible to avoid the occurrence of an unwanted pregnancy after unprotected or poorly protected sexual intercourse, i.e. in the absence of another contraceptive method or in the case of incorrect use or failure of the contraceptive method used. Clinical studies have shown that ulipristal acetate, administered in a single dose of 30 mg, is safe and effective for preventing an unwanted pregnancy after unprotected or poorly protected sexual intercourse (emergency contraception) when it is administered within 72 or 120 hours after sexual intercourse (Creinin et al., Obstetrics and Gynecology, 2006, 108(5): 1089-1097; Glasier et al., Lancet. 2010, 375(9714):555-62; Fine et al., Obstet Gynecol. 2010, 115:257-63).

Ulipristal acetate is also developed for on-demand contraception (International patent application WO 2010/119029). “On-demand” contraception makes it possible to avoid the occurrence of an unwanted pregnancy by administration of ulipristal acetate only when it is necessary, i.e. when sexual intercourse is expected or when it has just taken place, said administration of ulipristal acetate being potentially repeated during the menstrual cycle.

Certain non-steroidal anti-inflammatory compounds (NSAIDs) have also been proposed as contraceptives. Indeed, studies have shown that non-steroidal anti-inflammatories play a role in reducing or preventing ovulation in various animal species (rat, rabbit, sheep, monkey) (Zanagnolo et al., Fertility and Sterility, 1996, 65: 1036-1043; Murdoch, Prostaglandins, 1996, 52: 497-506). Furthermore, it has been shown that non-steroidal anti-inflammatories can block ovulation in women (Killick and Elstein, Fertility and Sterility, 1987, 47: 773-777). More specifically, other studies have shown that specific inhibitors of cyclooxygenases of type 2 delay follicular rupture and therefore ovulation in women (Pall et al., Human Reproduction, 2001, 16: 1323-1328; Bata et al., Journal of Clinical Pharmacology, 2006, 46(8): 925-932; Jesam et al., Human Reproduction, 2010, 25(2): 368-373). Studies in rodents and cattle have shown that prostaglandin production at the time of ovulation, under the impulse of COX-2 expressed in the granulosa cells, results in an increase in vascular permeability, and in the expression and activation of proteolytic enzymes required for oocyte expulsion (Richards J S, 1994, Endocr Rev, 15: 725-751; Richards J S, et al. 1995, Recent Prog Horm Res, 50: 223-254).

### SUMMARY OF THE INVENTION

The inventors now propose to combine at least one SPRM, which is preferably ulipristal acetate or a metabolite thereof, with at least one non-steroidal anti-inflammatory. This combination offers a better efficiency of ovulation inhibition.

An object of the invention is therefore a pharmaceutical composition comprising at least one SPRM, which is preferably ulipristal acetate or a metabolite thereof, and at least one non-steroidal anti-inflammatory compound, which is preferably a cyclooxygenase inhibitor.

Another object of the invention is a method of contraception in women, comprising administering a combination of at least one SPRM, which is preferably ulipristal acetate or a metabolite thereof, with at least one non-steroidal anti-inflammatory compound, which is preferably a cyclooxygenase inhibitor, preferably an oxicam, more preferably piroxicam.

According to a first embodiment, the SPRM, which is preferably ulipristal acetate or a metabolite thereof, and the non-steroidal anti-inflammatory compound are intended to be administered separately, simultaneously or sequentially.

According to another object, the SPRM, which is preferably ulipristal acetate or a metabolite thereof, and the non-steroidal anti-inflammatory compound are combined within the same pharmaceutical composition.

Another object of the invention is a kit, preferably a contraceptive kit, comprising, within the same packaging:
a pharmaceutical composition A comprising at least one SPRM, which is preferably ulipristal acetate or a metabolite thereof, in a physiologically acceptable medium; and
a pharmaceutical composition B comprising at least one non-steroidal anti-inflammatory compound.

### FIGURES

FIG. 1 shows the effect of the administration of piroxicam in mice having undergone an ovarian hyperstimulation treatment (superovulation). The mice are treated either with a control vehicle (V), or with piroxicam administered at 3 mg/kg (Prx 3 mg), 10 mg/kg (Prx 10 mg) or 30 mg/kg (Prx 30 mg), 8 hours after the injection of chorionic gonadotropin hormone (hCG). The oocytes released are counted 18 hours after the hCG injection. The diagram represents, for each group, the number of oocytes released, expressed as percentages relative to the control group (V). The percentages are represented as mean percentages±SEM (standard error of the mean). The notation * indicates that the difference observed compared with the control group is statistically significant (p<0.05).

FIG. 2 shows the effect of the administration of ulipristal acetate, alone or in combination with piroxicam, in mice having undergone an ovarian-hyperstimulation treatment. The experimental protocol is identical to that presented above. The experimental groups are the following: V: control group administered with the control vehicle; UPA: group administered with 40 mg/kg of ulipristal acetate; UPA+Prx 3 mg: group administered with 40 mg/kg of ulipristal acetate+3 mg/kg of piroxicam; UPA+Prx 30 mg: group administered with 40 mg/kg of ulipristal acetate+30 mg/kg of piroxicam. The diagram represents, for each group, the mean number of oocytes released, expressed as percentages (±SEM) compared with the control group (V). The notation * indicates that the difference observed compared with the control group is statistically significant (p<0.05).

FIG. 3 shows the ovarian histological sections, after staining with hematoxylin and eosin, originating from a mouse belonging to the control group (V), a mouse administered with 40 mg/kg of UPA (UPA), a mouse administered with 40 mg/kg of ulipristal acetate+3 mg/kg of piroxicam (UPA+Piroxicam 3 mg/kg), and a mouse administered with 40 mg/kg of ulipristal acetate+30 mg/kg of piroxicam (UPA+Piroxicam 30 mg/kg). CL: yellow body (corpus luteum), UF: unruptured follicle.

FIG. 4 shows the effect of the administration of meloxicam in mice having undergone an ovarian hyperstimulation treatment. The experimental groups are as follows: V: control group; Mlx 3 mg: group administered with 3 mg/kg of meloxicam; Mlx 10 mg: group administered with 10 mg/kg of meloxicam; Mlx 30 mg: group administered with 30 mg/kg of meloxicam. The diagram represents, for each group, the mean number of oocytes released, expressed as percentages (±SEM) compared with the control group (V). The notation * indicates that the difference observed as compared to the control group is statistically significant (p<0.05).

FIG. 5 shows the effect of the administration of ulipristal acetate, alone, or in combination with meloxicam, in mice having undergone an ovarian hyperstimulation treatment. The experimental groups are as follows: V: control group; UPA: group administered with 40 mg/kg of ulipristal acetate; UPA+Mlx 3 mg: group administered with 40 mg/kg of ulipristal acetate+3 mg/kg of meloxicam; UPA+Mlx 30 mg: group administered with 40 mg/kg of ulipristal acetate+30 mg/kg of meloxicam. The diagram represents, for each group, the mean number of oocytes released, expressed as percentages (±SEM) as compared to the control group (V). The notation * indicates that the difference observed as compared to the control group is statistically significant (p<0.05).

FIG. 6 shows the effect of the administration of ibuprofen in mice having undergone an ovarian hyperstimulation treatment. The experimental groups are the following: V: control group; Ibu 15 mg: group administered with 15 mg/kg of ibuprofen; Ibu 45 mg: group administered with 45 mg/kg of ibuprofen; Ibu 150 mg: group administered with 150 mg/kg of ibuprofen. The diagram represents, for each group, the mean number of oocytes released, expressed as percentages (±SEM) compared with the control group (V). The statistical study showed that the results obtained for the groups treated with ibuprofen are not significantly different from the results of the control group.

FIG. 7 shows the effect of the administration of ulipristal acetate, alone or in combination with ibuprofen, in mice having undergone an ovarian hyperstimulation treatment. The experimental groups are as follows: V: control group; UPA: group administered with 40 mg/kg of ulipristal acetate; UPA+Ibu 15 mg: group administered with 40 mg/kg of ulipristal acetate+15 mg/kg of ibuprofen; UPA+Ibu 150 mg: group administered with 40 mg/kg of ulipristal acetate+150 mg/kg of ibuprofen. The diagram represents, for each group, the mean number of oocytes released, expressed as percentages (±SEM) compared with the control group (V). The notation * means that the difference observed compared with the control group is statistically significant (p<0.05).

FIG. 8 shows the effect of the combined administration of UPA and a non-steroidal anti-inflammatory (NSAID) compared with the sole administration of UPA in mice having undergone an ovarian hyperstimulation treatment. UPA: group administered with 40 mg/kg of ulipristal acetate; UPA+Ibu 150 mg: group administered with 40 mg/kg of ulipristal acetate+150 mg/kg of ibuprofen; UPA+Mlx 30 mg: group administered with 40 mg/kg of ulipristal acetate+30 mg/kg of meloxicam; and UPA+Prx 30 mg: group administered with 40 mg/kg of ulipristal acetate+30 mg/kg of peroxicam. The diagram represents, for each group, the mean number of oocytes released, expressed as percentages (±SEM) as compared to the group administered with 40 mg/kg of UPA. The statistical analysis showed that the UPA-NSAID combinations are significantly more effective for inhibiting follicular rupture than the sole administration of UPA (p<0.05).

### DETAILED DESCRIPTION OF THE INVENTION

The efficacy of ulipristal acetate in emergency contraception is based on its capacity to inhibit or delay follicular rupture. Brache et al. (Hum Reprod, 2011, 9: 2256-2263) have shown that a single administration of EllaOne® (ulipristal acetate 30 mg) occurring after LH (luteinizing hormone) production has begun to increase, but before the LH peak, results in an inhibition of follicular rupture during the 5 days following administration in 78.6% of cases. This efficacy drops to 8.3% if the administration of ulipristal acetate occurs after the LH peak. As is shown in detail in the examples, the combined administration of a non-steroidal anti-inflammatory agent, in particular a cyclooxygenase inhibitor, and of ulipristal acetate (UPA) enables to increase, and even to potentiate, the capacity of UPA to inhibit follicular rupture.

On this basis, the inventors propose combining a SPRM with a non-steroidal anti-inflammatory, and thus providing a new method of contraception, whether it is an emergency contraception or a regular contraception, which displays a higher efficacity to inhibit ovulation.

### Progesterone Receptor Modulators:

The invention uses progesterone receptor modulators, preferably a selective progesterone receptor modulator (SPRM). The term “selective progesterone receptor modulator” is intended to mean a progesterone receptor ligand which exerts an agonist activity, an antagonist activity or a mixed agonist/antagonist activity in a tissue-specific manner, preferably an agonist or mixed agonist/antagonist activity. From his general knowledge, a skilled artisan can determine, by means of routine experiments, whether a compound is an SPRM, in particular by referring to the articles by Smith and O'Malley, Endocrine Review, 25(1):45-71, and by Chabbert-Buffet et al., Human Reproduction Update, 2005, 11, 293-307.

The SPRM compound may be a non-steroidal compound or a steroid derivative. Examples of non-steroidal selective progesterone receptor modulators are given in the following publications: Dong et al., Steroids, 2004, 69:201-207, Zhi et al., J Med Chem, 2003, 46:4104-4112 and Zhi et al., Curr Top Med Chem, 2008, 8:766-780, the content thereof being incorporated by way of reference.

In one preferred embodiment, the SPRM compound is a steroidal derivative. It may be selected from steroidal derivatives substituted on 11β position with an aryl group. Suitable aryl groups comprise, without being limited to, 4-(dimethylamino)phenyl, 4-acetylphenyl, and benzaldoxime.

As a matter of interest, the carbon atoms of the steroid core are numbered in the following way:

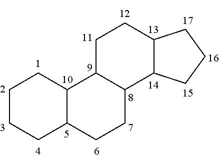 [SMILES: C1CCC2C(C1)CCC1C3CCCC3CCC21]

Examples of steroidal SPRMs are given in the following publications: Rao et al., Steroids, 1998, 63:523-530 and Chabbert-Buffet et al., Human Reproduction Update, 2005, 11, 293-307. In particular, Chabbert-Buffet et al. cite mifepristone, onapristone, asoprisnil, ulipristal acetate, Org 33628 and Org 31710 as being SPRMs.

In certain embodiments, the SPRM is a steroid selected from the group of SPRM compounds of formula (I) below:

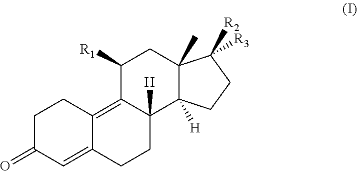

wherein:
R₁ represents an aryl group optionally substituted with one or more groups independently in the ortho, para or meta position, preferably selected from the group consisting of H, OH, —CH═N—OH, —C(═O)—R₄, a C₁-C₅ alkoxy group, a C₁-C₅ alkylamine group or a C₁-C₅ dialkylamine group,
R₂ represents —OH, a C₁-C₅ alkoxy group or —C(═O)—R₄, and
R₃ represents —OH, a C₁-C₅ alkoxy group, a C₂-C₅ alkynyl, a C₂-C₅ alkenyl or —O—C(═O)—R₄,
R₄ being selected from a C₁-C₃ alkyl group and a C₁-C₅ alkoxy group,
   and metabolites and pharmaceutically acceptable salts thereof.

Preferably, R₃ represents —OH, a C₁-C₅ alkoxy group, a C₂-C₅ alkynyl or —O—C(═O)—R₄.

A C₁-C₃ alkyl encompasses methyl, ethyl, propyl and isopropyl groups.

A C₁-C₅ alkoxy group encompasses the groups of formula —(CH₂)ₙO(CH₂)₍₄₋ₙ₎CH₃, n being an integer ranging from 0 to 4.

In a preferred embodiment, the SPRM is selected from the group of SPRM compounds of formula (Ia) below, et also metabolites and pharmaceutically acceptable salts thereof:

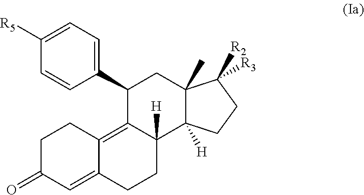

wherein:
R₅ represents:
   —NR₆R₇ wherein R₆ and R₇ represent, independently to each other, —H or a C₁-C₃ alkyl, R₆ and R₇ preferably being selected from H and —CH₃;
   —CH═N—OR₈ wherein R₈ represents —H or —C(═O)—X—R₉ with R₉ being a C₁-C₃ alkyl and X representing O, NH or S; or
   —C(═O)R₁₀ wherein R₁₀ represents a C₁-C₃ alkyl,
R₂ and R₃ being as previously defined.

Preferably, R₃ represents —OH, a C₁-C₅ alkoxy group, a C₂-C₅ alkynyl or —O—C(═O)—R₄ with R₄ being selected from a C₁-C₃ alkyl group and a C₁-C₅ alkoxy group.

Such compounds comprise, without being limited to, mifepristone, ulipristal acetate, asoprisnil and telapristone. In particular, mifepristone corresponds to the compound of formula (Ia) wherein R₅ is —N(CH₃)₂, R₂ is OH and R₃ is —C≡C—CH₃.

In a preferred embodiment, the SPRM is selected from the group of SPRM compounds of formula (Ia) wherein:
R₂ represents —OH, —OCH₃, —C(═O)CH₃ or —(C═O)—CH₂—O—CH₃,
R₃ represents —CH₂—O—CH₃ or —O—C(═O)—CH₃,
R₅ represents —NH₂, —NHCH₃, —N(CH₃)₂ or —CH═N—OR₈ wherein R₈ represents H, —(C═O)—S—C₂H₅ or —C(═O)—NH—C₂H₅.

In certain embodiments, the SPRM is selected from the compounds of formula (Ia) wherein R₅ is —CH═NOR₈. Such compounds encompass:
Asoprisnil (R₈ is H, R₂ is OMe and R₃ is —CH₂OMe),
J912 (R₈ is H, R₂ is H and R₃ is —CH₂OMe),
J956 (also known as Asoprisnil ecamate) (R₈ is —C(═O)—NH—C₂H₅, R₂ is —OMe and R₃ is —CH₂OMe), and
J1042 (R₈ is —C(═O)S—C₂H₅, R₂ is —OCH₃ and R₃ is —CH₂OMe).

In other embodiments, the SPRM is selected from the compounds of formula (Ia) wherein R₅ is —N(Me)₂. Such compounds encompass ulipristal acetate (R₂ is —C(═O)—CH₃ and R₃ is —O—C(═O)CH₃) and telapristone (Proellex® also known as CDB-4124) (R₂ is —C(═O)—CH₂—O—CH₃ and R₃ is —O—C(═O)—CH₃).

In a preferred embodiment, the SPRM compound is ulipristal acetate (also known as CDB-2914).

Ulipristal acetate is 17α-acetoxy-11β-[4-N,N-dimethylaminophenyl)-19-norpregna-4,9-diene-3,20-dione (IUPAC nomenclature), represented by formula I:

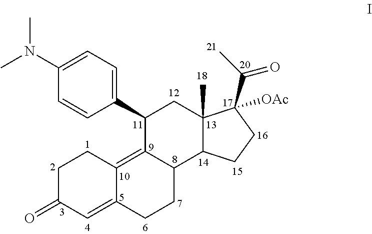 [SMILES: CC(=O)O[C@]1(C(C)=O)CCC2C3CCC4=CC(=O)CCC4=C3[C@@H](c3ccc(N(C)C)cc3)C[C@@]21C]

This compound and the processes for obtaining this compound are described in U.S. Pat. No. 4,954,490; U.S. Pat. No. 5,073,548 and U.S. Pat. No. 5,929,262 and International applications WO 2004/065405 and WO 2004/078709 in particular.

One may also use one of the metabolites of ulipristal acetate, as described in Attardi et al., Journal of Steroid Biochemistry and Molecular Biology, 2004, 88: 277-288. The metabolites of ulipristal acetate encompasses, inter alia:
the compound CDB-3877 (compound of formula (Ia) wherein R₅ is —NHCH₃, R₂ is —C(═O)—CH₃ and R₃ is —O—C(═O)—CH₃) and
the compound CDB-3963 (compound of formula (Ia) wherein R₅ is —NH₂, R₂ is —C(═O)—CH₃ and R₃ is —O—C(═O)—CH₃.
The metabolites of ulipristal acetate which are described by Attardi et al. are as follows:

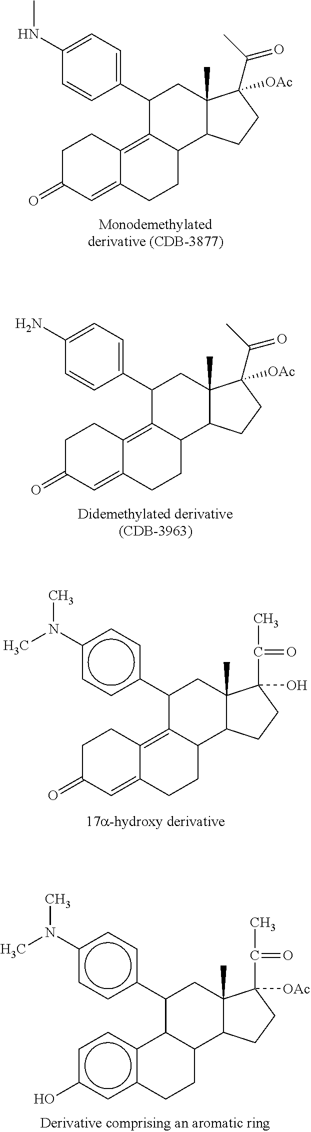

The metabolite of ulipristal acetate is preferably CDB-3877 or CDB-3963, even more preferably CDB-3877.

### Non-Steroidal Anti-Inflammatory Compounds:

The term “NSAID” refers to any non-steroidal anti-inflammatory compound. These compounds are classified according to their capacity to inhibit a cyclooxygenase. Cyclooxygenases 1 and 2 are the two main isoforms and most NSAIDs are mixed inhibitors of the two isoforms. Five categories of mixed inhibitors are generally distinguished: (1) propionic acid derivatives, for instance ibuprofen, naproxen, naprosyn, diclofenac and ketoprofen; (2) acetic acid derivatives, for instance tolmetin and slindac; (3) fenamic acid derivatives, for instance mefenamic acid and meclofenamic acid; (4) biphenylcarboxylic acid derivatives, for instance diflunisal and flufenisal; and (5) oxicams, for instance meloxicam, piroxicam, sudoxicam and isoxicam.

Examples of COX-2 selective inhibitor compounds include celecoxib (SC-58635), DUP-697, flosulide (CGP-28238), meloxicam, 6-methoxy-2-naphthylacetic acid (6-MNA), rofecoxib, MK-966, nabumetone, nimesulide, NS-398, SC-5766, SC-58215, and T-614.

In certain embodiments of the invention, the non-steroidal anti-inflammatory compound is selected from oxicams, propionic acid derivatives, preferably 2-arylpropionic acid derivatives, and combinations thereof.

Thus, the non-steroidal anti-inflammatory compound can be selected from oxicams. The oxicams comprise in particular piroxicam, tenoxicam, droxicam, lornoxicam, meloxicam and sudoxicam, and combinations thereof.

The non-steroidal anti-inflammatory compound may also be selected from propionic acid derivatives which comprise, among others, ibuprofen, naproxen, naprosyn, diclofenac and ketoprofen.

Preferably, the non-steroidal anti-inflammatory agent is selected from meloxicam and piroxicam, and combinations thereof. Alternatively, in the invention, the non-steroidal anti-inflammatory compound is a cyclooxygenase type 2 (COX-2) inhibitor selected from the group consisting of indomethacin, naproxen, ibuprofen, acetominophen, meloxicam, piroxicam, etodolac and celecoxib. Derivatives of these compounds are also included, for instance naproxinod, which is composed of naproxen and also of a nitric oxide molecule.

For the purposes of the invention, a cyclooxygenase type 2 (COX-2) inhibitor may be a selective or non-selective COX-2 inhibitor.

Preferably, a non-selective COX inhibitor, i.e. a compound which inhibits both COX-1 and COX-2 in a non-selective way is used.

Thus, piroxicam or ibuprofen is preferably used as COX inhibitor.

Selective COX-2 inhibitors, for instance in particular meloxicam, can also be used.

### Pharmaceutical Compositions:

The SPRM, which is preferably ulipristal acetate or a metabolite thereof, and the NSAID can be combined within the same pharmaceutical composition, or be used in the form of separate pharmaceutical compositions which can be administered simultaneously or sequentially. In particular, they can be administered separately, namely either concomitantly, or independently, for example, with a time shift.

In accordance with the invention, the SPRM, which is preferably ulipristal acetate or a metabolite thereof, and the NSAID are used in combination in order to potentiate the effects of the ulipristal acetate on ovulation inhibition.

Whatever the route of administration and the form of the pharmaceutical compositions, the compounds are preferably administered in amounts that are synergistic with respect to the anti-ovulation effect.

The pharmaceutical compositions according to the invention advantageously comprise one or more pharmaceutically acceptable excipients or vehicles. One may cite, for example, buffered, isotonic, physiological, etc. saline solutions, compatible with pharmaceutical use and known to those skilled in the art. The compositions may contain one or more agents or vehicles selected from dispersants, solubilizers, stabilizers, preservatives, etc. Agents or vehicles which can be used (liquid and/or injectable and/or solid) formulations are in particular methylcellulose, hydroxymethylcellulose, carboxymethylcellulose, polysorbate 80, mannitol, gelatin, lactose, vegetable oils, acacia, etc. The compositions may be formulated in the form of injectable suspensions, gels, oils, tablets, suppositories, powders, gelatine capsules, capsules, etc., optionally by means of galenic forms or devices providing prolonged and/or delayed release. For this type of formulation, one may advantageously use an agent such as cellulose, carbonates or starches.

The combination according to the invention can be administered by any appropriate route of administration, for example via the oral, buccal, sublingual, vaginal, intrauterine, rectal, or transdermal route or via the parenteral route, for example by intravenous, intracutaneous or intradermal injection. Preferably, oral administration is envisaged.

Consequently, the medicament may be made up in the form of tablets, gel capsules, powder or any form for a solid oral preparation or in any form of oral preparation. The pharmaceutical composition generally comprises a physiologically acceptable medium, for example for the preparation of tablets or of gel capsules or for an oral preparation, such as the vehicles used entirely conventionally.

Particular ulipristal acetate tablets are, for example, described in patent application WO 2010/066749.

Preferably, the SPRM, which is preferably ulipristal acetate or a metabolite thereof, is used at a dosage of from 1 mg to 100 mg, 2.5 to 100 mg, preferably from 5 to 50 mg, preferably 5 to 30 mg, 15 mg to 35 mg, or from 10 to 30 mg. The NSAID is itself advantageously used at a dosage of from 5 mg to 1 g per intake, preferably from 20 to 800 mg per intake, preferably from 20 to 400 mg, more preferably from 100 to 200 mg per intake, preferably in one daily intake. It is understood that certain NSAIDs, such as ibuprofen, may be used at doses of from 200 to 600 mg approximately. Piroxicam may be advantageously used at a dosage of from 20 to 80 mg. Meloxicam may be advantageously used at a dosage of from 5 to 60 mg. Preferably, the SPRM, which is preferably ulipristal acetate or a metabolite thereof, and the NSAID are used in a ratio of from 0.01 to 10, preferably from 5/80 to 30/20, more preferably of approximately 0.05/2.

Preferentially, the pharmaceutical combination according to the invention comprises approximately 30 mg of ulipristal acetate or of a metabolite thereof, and approximately 5 to 400 mg of one or more non-steroidal anti-inflammatories. Such a pharmaceutical combination is preferentially intended for emergency or on-demand contraception.

In regular contraception, it is preferable to use a pharmaceutical combination comprising from 1 mg to 5 mg of ulipristal acetate.

The pharmaceutical compositions, intended to be administered simultaneously, separately or sequentially, can also be provided in kit form, for example in the form of a blister pack of contraceptive pills.

Thus, a kit, preferably a contraceptive kit, can be produced within the same packaging and can comprise:
a pharmaceutical composition A comprising an SPRM, which is preferably ulipristal acetate or a metabolite thereof, in a physiologically acceptable medium; and
a pharmaceutical composition B comprising one or more non-steroidal anti-inflammatories in a physiologically acceptable medium.

The contraceptive kit according to the invention may comprise one or more dose units of the pharmaceutical composition A and one or more dose units of the pharmaceutical composition B. The number of dose units of each composition depends on the selected dosage regimen and on the contraceptive indication which is sought, i.e. regular contraception, on-demand contraception or emergency contraception, as explained in detail hereinafter.

### Indications:

The combination of an SPRM, which is preferably ulipristal acetate or a metabolite thereof, in combination with one or more non-steroidal anti-inflammatories is useful in a method of contraception in women.

The terms “method of contraception in women” and “contraception in women” refer to a method for avoiding the occurrence of an unwanted pregnancy, in a woman of child-bearing age, during sexual intercourse.

Preferentially, the invention relates to a method of emergency contraception in women.

The terms “method of emergency contraception in women” and “emergency contraception in women” refer to a method for avoiding the occurrence of an unwanted pregnancy, in a woman of child-bearing age, after unprotected or poorly protected sexual intercourse, i.e. in the absence of contraception or in the case of failure of the contraceptive method used.

The invention also relates to a method of regular contraception in women. If the SPRM is administered orally, the method of contraception may, for example, consist of a daily administration for at least 20 days in the cycle, or every day without interruption.

Moreover, the invention relates to a method of on-demand contraception in women.

The terms “method of on-demand contraception in women” and “on-demand contraception in women” refer to a method for avoiding the occurrence of an unwanted pregnancy, in a woman of child-bearing age, by administering the treatment when it is necessary, i.e. when sexual intercourse is expected or when it has just occurred.

The method of emergency contraception in women according to the invention comprises the administration, to a female subject of child-bearing age, of an effective amount of SPRM, preferably of ulipristal acetate or a metabolite thereof, in combination with one or more non-steroidal anti-inflammatories, after unprotected or poorly protected sexual intercourse. In certain cases, such as when the SPRM is ulipristal acetate, the administration may be carried out up to approximately 5 days after an unprotected or poorly protected sexual intercourse. Preferably, the combination of the invention is administered within 120 h, preferably 72 h, preferably 48 h, preferably 24 h after sexual intercourse.

The method of on-demand contraception in women according to the invention comprises administering a female subject of child-bearing age, with an effective amount of SPRM, preferably of ulipristal acetate or of a metabolite thereof, in combination with one or more non-steroidal anti-inflammatories, approximately 3 days, preferably within 24 h, preferably within 12 h, before unprotected sexual intercourse and approximately 5 days, preferably within 120 h, preferably 72 h, preferably 48 h, preferably 24 h, after unprotected sexual intercourse. The administration can be repeated at least once a week, or even several times per month, or several times per week.

Such a method of “on-demand” contraception can replace a method of conventional regular hormonal contraception.

In the methods of contraception according to the invention, the administrations of the SPRM and of the non-steroidal anti-inflammatory agent may be simultaneous, sequential or separate, i.e. spread out over time. The administration of the SPRM can thus precede or follow by several minutes or even several hours the administration of the NSAID. Preferably, the administration of the SPRM and that of the NSAID are simultaneous or separated over time by at most 12 hours, by at most 6 hours, by at most 4 hours, by at most 3 hours, by at most 2 hours and preferably by at most 1 hour.

In the methods of emergency or on-demand contraception according to the invention, the administration of the non-steroidal anti-inflammatory agent may be repeated one or more times, over the course of one or more days, after its administration in combination with the SPRM. For example, the administration of the NSAID can be repeated 1 to 5 times at a rate of one dose per day.

By way of non-limiting example, the method of emergency contraception according to the invention may comprise the combined administration of the SPRM and of the NSAID simultaneously or in a manner separated over time by at most 6 hours, preferably by at most 1 hour, and optionally the administration of a daily dose of NSAID over the course of 1 to 5 days following said combined administration of the SPRM and of the NSAID.

In one preferred embodiment, the administration of the NSAID is not repeated.

A object of the present invention is also a selective progesterone receptor modulator (SPRM), preferably ulipristal acetate or a metabolite thereof, for use as a contraceptive in women in combination with at least one non-steroidal anti-inflammatory compound.

An additional object of the invention is a combination product comprising an SPRM, preferably ulipristal acetate or a metabolite thereof, and at least one non-steroidal anti-inflammatory compound, for separate, simultaneous or sequential use in contraception in women.

Finally, an additional object according to the invention is the use of an SPRM, preferably ulipristal acetate or a metabolite thereof, and of at least one non-steroidal anti-inflammatory compound, for the manufacture of a medicament intended for contraception in women.

The medicament may be in the form of one or more dose units, each dose unit comprising both the SPRM and the non-steroidal anti-inflammatory compound. By way of an alternative, the medicament comprises at least one dose unit comprising the SPRM and at least one dose unit comprising the non-steroidal anti-inflammatory compound.

Preferably, these various aspects of the invention refer to emergency contraception. It goes without saying that the particular embodiments of these various aspects of the invention are as described previously for the composition, the kit and the contraception method according to the invention.

The purpose of the examples hereinafter is to illustrate the invention without however limiting the scope thereof.

### Examples

The effect of the combined administration of ulipristal acetate (UPA) and of a non-steroidal anti-inflammatory agent (NSAID) on LH-induced ovarian follicle rupture was evaluated in an animal model of ovarian hyperstimulation (superovulation model).

a) Inhibitory Effect of Piroxicam in Combination with Ulipristal Acetate on Ovulation

24 to 28-day-old immature CD1 mice were subjected to ovarian hyperstimulation by injection of 5 IU of PMSG (pregnant mare's serum gonadotropin) followed, 48 h later, by 5 IU of hCG (chorionic gonadotropin hormone) intraperitoneally. The mice were divided up into several groups, each of 10 mice. 8 hours after the hCG injection, the mice were administered, intraperitoneally, with either the compound(s) to be tested, or the placebo (vehicle). The mice were euthanized 18 hours after the hCG injection. The effect of the compound(s) on follicular rupture and therefore ovulation was determined by counting the oocytes released into the oviducts. In experiment series No. 2, a morphological and histological examination of the ovaries was carried out so as to evaluate the presence of unruptured follicles and/or of yellow bodies in the ovaries of each group of animals.

Presented below are the experiment series carried out and the doses administered to each group of mice.

Series 1:
Group 1: placebo
Group 2: 3 mg/kg of piroxicam
Group 3: 10 mg/kg of piroxicam
Group 4: 30 mg/kg of piroxicam.

Series 2:
Group 1: placebo
Group 2: 40 mg/kg of ulipristal acetate
Group 3: 40 mg/kg of ulipristal acetate+3 mg/kg of piroxicam
Group 4: 40 mg/kg of ulipristal acetate+30 mg/kg of piroxicam.

### Results:

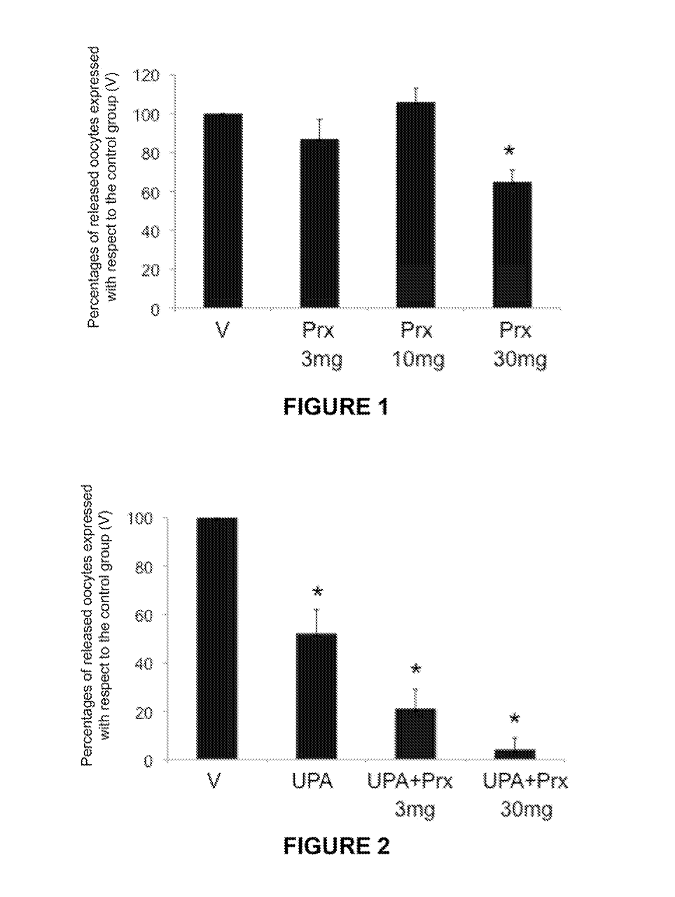

FIG. 1 shows the number of oocytes released into the oviducts for the various groups of mice of the first series of experiments, expressed as mean percentages compared with the control group 1. While the administration of 3 mg or 10 mg of piroxicam does not significantly reduce the number of oocytes released into the oviducts (and therefore ovulation), the administration of 30 mg of piroxicam enables to significantly reduce the number of ovulations observed in the mice of group 4 as compared to the control group.

FIG. 2 shows the results of counting the oocytes harvested in the oviducts for the mice of the various groups of experiment series 2. The mean number of oocytes released in the group of mice administered with 40 mg/kg of UPA 8 hours after the hCG injection was significantly reduced (approximately −50%) as compared to the control group of mice. The mean number of oocytes released in the groups of mice which received 40 mg/kg of UPA+3 mg/kg or 30 mg/kg of piroxicam is significantly lower than the number of oocytes released in the control group and in group 2. Notably, a dose of 3 mg/kg of piroxicam potentiates the ovulation inhibition effect of UPA, whereas no significant effect on ovulation was detected at this dose of piroxicam (and in the absence of UPA) (see group 2 of experiment series 1). This demonstrates a synergistic effect on the inhibition of follicular rupture resulting from the combination of UPA and piroxicam.

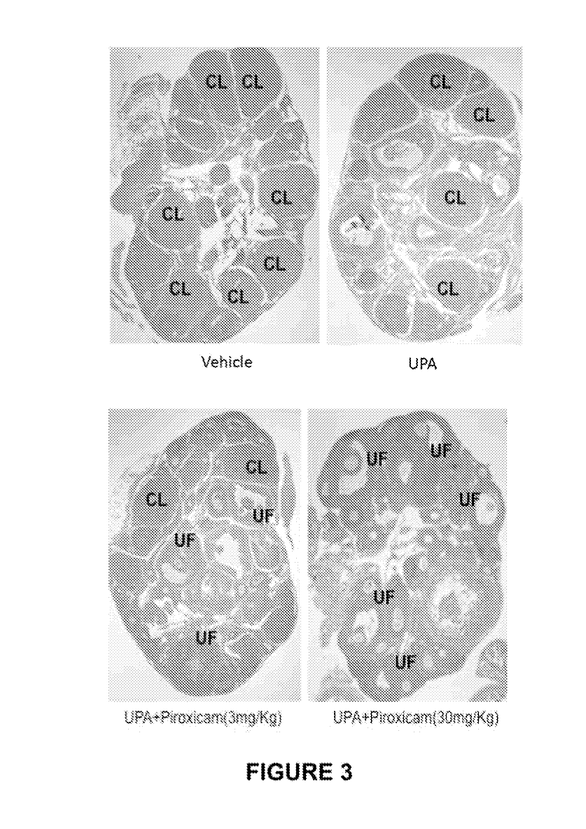

FIG. 3 shows the histological sections of ovaries for mice of each group of mice of series 2. Notably, the histological sections for groups 3 and 4 reveal a large number of unruptured ovarian follicles and very few, or even no, yellow bodies (corpus luteum). The yellow bodies are more numerous in the mice administered with UPA in the absence of piroxicam. The ovarian histological sections of the control group demonstrate the presence of numerous yellow bodies. These histological observations are coherent with the results relating to the number of follicles released and confirm that the UPA-piroxicam combination inhibits or delays ovulation, more effectively than the administration of UPA in the absence of NSAID.

b) Inhibitory Effect of Meloxicam in Combination with Ulipristal Acetate on Ovulation

A protocol similar to that used for the piroxicam-ulipristal acetate combination was implemented in order to evaluate the ovulation inhibition effect of the meloxicam-ulipristal acetate combination.

The following series of experiments were carried out:

Series 1:
Group 1: placebo
Group 2: 3 mg/kg of piroxicam
Group 3: 10 mg/kg of piroxicam
Group 4: 30 mg/kg of piroxicam.

Series 2:
Group 1: placebo
Group 2: 40 mg/kg of ulipristal acetate
Group 3: 40 mg/kg of ulipristal acetate+3 mg/kg of piroxicam
Group 4: 40 mg/kg of ulipristal acetate+30 mg/kg of piroxicam.

### Results:

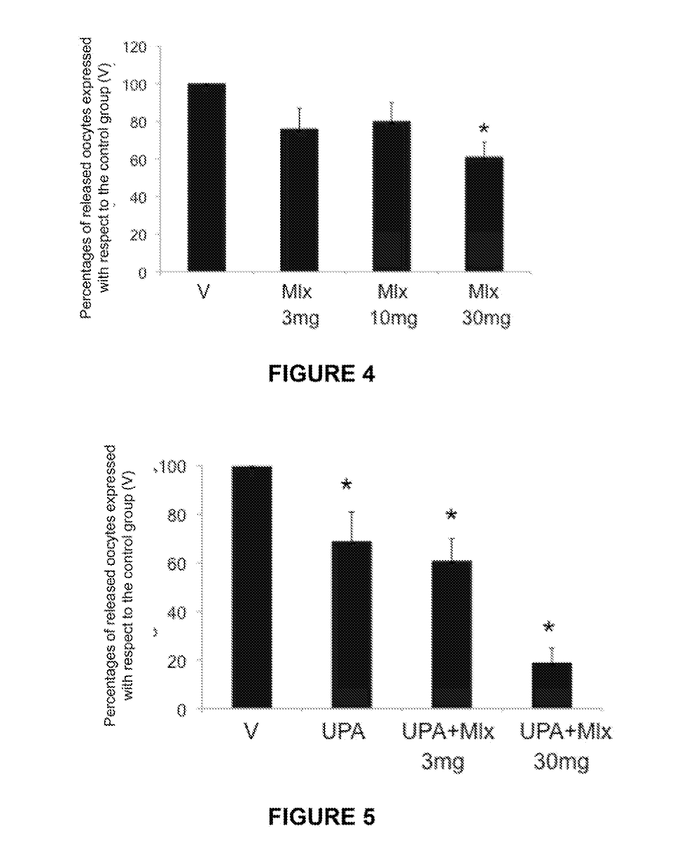

FIGS. 4 and 5 show the results obtained for experiment series 1 and 2, respectively.

FIG. 4 shows that meloxicam, when it is administered alone, only weakly inhibits ovulation. A significant effect compared to the control group is observed only for the administration of a dose of 30 mg of meloxicam. FIG. 5 shows that the administration of meloxicam in combination with UPA makes it possible to significantly reduce the number of oocytes released compared with the control group. The administration of 30 mg/kg of meloxicam very significantly potentiates the ovulation inhibition effect of UPA in the superovulation model studied. This clearly emerges from the comparison of the results obtained for group 2 with those obtained for group 4.

The results obtained demonstrate that cyclooxygenase inhibitors, such as the compounds belonging to the oxicam family, are capable of potentiating the inhibitory effect of UPA on ovulation.

c) Inhibitory Effect of Ibuprofen in Combination with Ulipristal Acetate on Ovulation

A protocol similar to that used to evaluate the effect of the piroxicam-ulipristal acetate combination was implemented in order to evaluate the ibuprofen-ulipristal acetate combination.

The following series of experiments were carried out:

Series 1:
Group 1: placebo
Group 2: 15 mg/kg of ibuprofen
Group 3: 45 mg/kg of ibuprofen
Group 4: 150 mg/kg of ibuprofen.

Series 2:
Group 1: placebo
Group 2: 40 mg/kg of ulipristal acetate
Group 3: 40 mg/kg of ulipristal acetate+15 mg/kg of ibuprofen
Group 4: 40 mg/kg of ulipristal acetate+150 mg/kg of ibuprofen.

### Results:

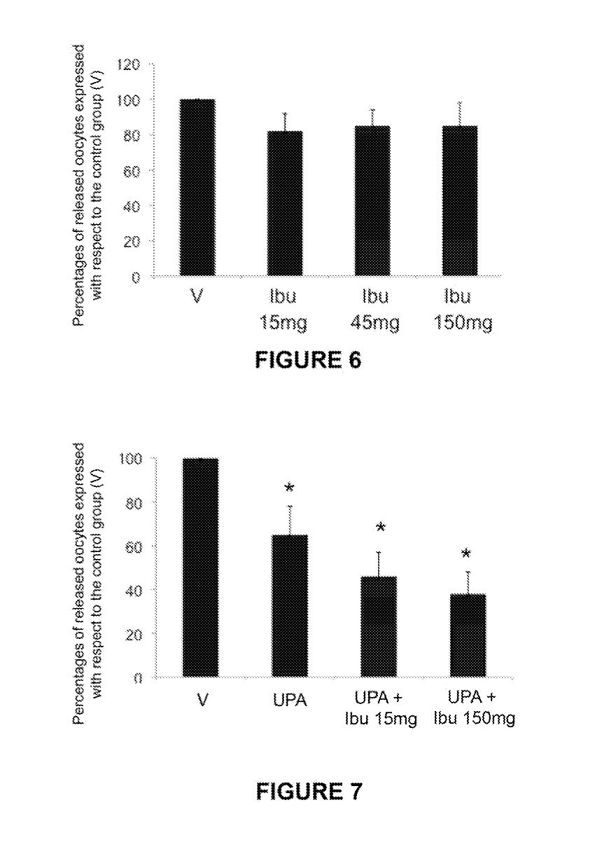

FIGS. 6 and 7 show the results obtained for experiment series 1 and 2, respectively.

FIG. 6 illustrates that the administration of a dose of between 15 mg and 150 mg of ibuprofen has no significant effect on ovulation in the ovary hyperstimulation model studied.

As illustrated by FIG. 7, a significant decrease in the number of oocytes released is observed in groups 2, 3 and 4 compared with the control group. The administration of the ulipristal acetate-ibuprofen combination is more effective than ulipristal acetate, administered alone, for inhibiting ovulation.

### CONCLUSION

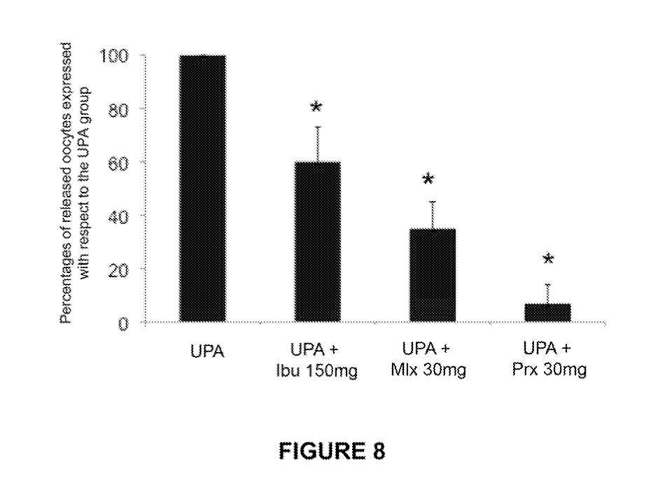

FIG. 8 summarizes the effect of UPA and the various UPA-NSAID combinations tested on ovulation inhibition in the ovarian hyperstimulation model studied. It appears that the combinations of UPA-NSAID are significantly more effective for preventing follicular rupture than UPA alone. Moreover, it emerges from the results presented above that the tested non-steroidal anti-inflammatory compounds enable to potentiate the inhibitory action of ulipristal acetate on follicular rupture, the NSAID and the UPA acting in synergy.

1. A pharmaceutical composition comprising at least one selective progesterone receptor modulator (SPRM) and at least one non-steroidal anti-inflammatory compound.

2. (canceled)

3. The pharmaceutical composition of claim 1, wherein the SPRM is a compound of formula 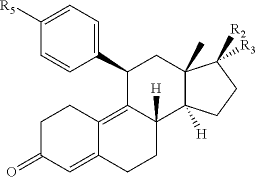
wherein:
R₂ represents —OH, a C₁-C₅ alkoxy group or —C(═O)—R₄,
R₃ represents —OH, a C₁-C₅ alkoxy group, a C₂-C₅ alkynyl, a C₂-C₅ alkenyl or —O—C(═O)—R₄,
R₄ being selected from a C₁-C₃ alkyl group and a C₁-C₅ alkoxy group, and
R₅ represents:
—NR₆R₇ wherein R₆ and R₇ are, independently to each other, —H or a C₁-C₃ alkyl, R₆ and R₇ preferably being selected from H and —CH₃;
—CH═N—OR₈ wherein R₈ represents —H or —C(═O)—X—R₉ with R₉ being a C₁-C₃ alkyl and X representing O, NH or S; or
—C(═O)R₁₀ wherein R₁₀ represents a C₁-C₃ alkyl,
or a metabolite or pharmaceutically acceptable salt thereof.

4. The pharmaceutical composition of claim 1, wherein the SPRM is ulipristal acetate or a metabolite thereof.

5. The pharmaceutical composition of claim 1, wherein the non-steroidal anti-inflammatory compound is a cyclooxygenase inhibitor.

6. The pharmaceutical composition of claim 1, wherein the non-steroidal anti-inflammatory compound is a selective or non-selective cyclooxygenase type 2 (COX-2) inhibitor.

7. The pharmaceutical composition of claim 5, wherein the cyclooxygenase inhibitor is an oxicam.

8. The pharmaceutical composition of claim 7, wherein the oxicam is piroxicam or meloxicam.

9. (canceled)

10. The pharmaceutical composition according to claim 1, wherein the non-steroidal anti-inflammatory compound is a cyclooxygenase type 2 (COX-2) inhibitor selected from the group consisting of indomethacin, naproxen, naproxinod, ibuprofen, acetominophen, etodolac and celecoxib.

11. The pharmaceutical composition of claim 1, which is for oral administration.

12-14. (canceled)

15. A method for providing contraception in a woman, comprising the step of administering at least one selective progesterone receptor modulator (SPRM), and at least one non-steroidal anti-inflammatory compound to the woman.

16. The method of claim 15, wherein the SPRM is ulipristal acetate or a metabolite thereof.

17. The method of claim 15, wherein the non-steroidal anti-inflammatory compound is piroxicam or meloxicam.

18. The method of claim 15, wherein the SPRM and the non-steroidal anti-inflammatory compound are administered separately, simultaneously or sequentially.

19. The method of claim 15, wherein the SPRM and the non-steroidal anti-inflammatory compound are combined within the same pharmaceutical composition.

20. The method of claim 15, used for providing emergency contraception or providing regular contraception.

21. (canceled)

22. A contraceptive kit, comprising, within the same packaging:
i. a pharmaceutical composition A comprising at least one SPRM; and
ii. a pharmaceutical composition B comprising at least one non-steroidal anti-inflammatory compound.

23. The method of claim 15, wherein the SPRM is a compound of formula: 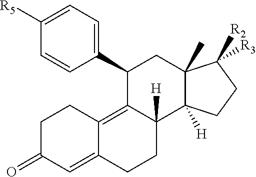
wherein:
R₂ represents —OH, a C₁-C₅ alkoxy group or —C(═O)—R₄, and
R₃ represents —OH, a C₁-C₅ alkoxy group, a C₂-C₅ alkynyl, a C₂-C₅ alkenyl or —O—C(═O)—R₄,
R₄ being selected from a C₁-C₃ alkyl group and a C₁-C₅ alkoxy group, and
R₅ represents:
—NR₆R₇ wherein R₆ and R₇ are, independently to each other, —H or a C₁-C₃ alkyl, R₆ and R₇ preferably being selected from H and —CH₃;
—CH═N—OR₈ wherein R₈ represents —H or —C(═O)—X—R₉ with R₉ being a C₁-C₃ alkyl and X representing O, NH or S; or
—C(═O)R₁₀ wherein R₁₀ represents a C₁-C₃ alkyl,
or a metabolite or pharmaceutically acceptable salt thereof.

24. The method of claim 15, wherein the non-steroidal anti-inflammatory compound is a selective or non-selective cyclooxygenase type 2 (COX-2) inhibitor.
